(19) United States
(12) Patent Application Publication
(10) Pub. No.: US 2016/0220720 A1
(43) Pub. Date: Aug. 4, 2016

(54) COMPOSITIONS COMPRISING POLYAMINE POLYMER COMPATIBLE PERFUME MATERIALS

(71) Applicant: The Procter & Gamble Company, Cincinnati, OH (US)

(72) Inventors: Prakash J MADHAV, Maineville, OH (US); Judith Ann HOLLINGSHEAD, Batavia, OH (US); Cahit EYLEM, West Chester, OH (US); Zaiyou LIU, West Chester, OH (US); Steven Anthony HORENZIAK, Cincinnati, OH (US)

(21) Appl. No.: 15/095,182

(22) Filed: Apr. 11, 2016

Related U.S. Application Data
(63) Continuation of application No. 14/046,221, filed on Oct. 4, 2013.

(51) Int. Cl. A61L 9/01; C11D 3/37; C11B 9/00; C11D 3/50

(52) U.S. Cl. CPC A61L 9/01; C11D 3/50; C11D 3/3769; C11B 9/0061

(57) ABSTRACT

Compositions comprising polyamine polymers and polyamine polymer compatible perfume materials, more specifically, perfume aldehydes that do not react with polyamine polymers are provided.

### FIELD OF THE INVENTION

The present invention relates to compositions comprising polyamine polymers and polyamine polymer compatible perfume materials, more specifically, perfume aldehydes that do not react with polyamine polymers.

### BACKGROUND OF THE INVENTION

Perfume aldehydes are commonly used in liquid phase applications such as fabric refreshers for their characteristic fresh scents. While they provide a fresh scent, these perfume aldehydes are also reactive with malodor reducing actives such as polyamine polymers, thus binding to such polyamine polymers and reducing malodor efficacy on treated surfaces or in the air.

To overcome this problem, formulators have avoided perfume aldehydes or added additional perfume masking materials/malodor reducing actives, such as cyclodextrin and/or metal salts, to formulations containing a polyamine polymer and perfume aldehydes.

There remains a need to provide a composition comprising a polyamine polymer and perfume aldehydes that reduces malodor and provides a fresh scent on treated surfaces or in the air without requiring the added cost of additional perfume masking materials or additional malodor reducing actives.

### SUMMARY OF THE INVENTION

In one embodiment, there is provided, a malodor control composition comprising about 0.001% to about 2%, by weight of said composition, of a polyamine polymer; and a perfume mixture comprising a perfume aldehyde component comprising less than about 85%, by weight of said perfume aldehyde component, of perfume aldehydes having an R value less than 1 wherein:

R=2.21+(0.159×nrbond)−(0.0559×rvalCar)+(14.4×xch6)+(0.240×CdssC);

wherein R is measured at pH 6-8 in an aqueous carrier.

In another embodiment, there is provided a malodor control composition comprising about 0.001% to about 2%, by weight of said composition, of a polyamine polymer. The composition also comprises a perfume mixture comprising: a perfume aldehyde component comprising at least one perfume aldehyde selected from the group consisting of: amyl cinnamic aldehyde, anisic aldehyde, benzaldehyde, citronellal, cuminic aldehyde, citronellal oxyacetaldehyde, floralozone, heliotropin, hexyl cinnamic aldehyde, and mixtures thereof; and less than about 60%, by weight of said perfume aldehyde component, of perfume aldehydes having an R value less than 1 wherein:

R=2.21+(0.159×nrbond)−(0.0559×rvalCar)+(14.4×xch6)+(0.240×CdssC);

wherein R is measured at pH 6-8 in an aqueous carrier.

In yet another embodiment, there is provided a method of reducing malodors on surfaces or in the air comprising the steps of: providing an effective amount of composition comprising about 0.001% to about 2%, by weight of said composition, of a polyamine polymer; and a perfume mixture comprising a perfume aldehyde component comprising less than about 85%, by weight of said perfume aldehyde component, of perfume aldehydes having an R value less than 1 wherein:

R=2.21+(0.159×nrbond)−(0.0559×rvalCar)+(14.4×xch6)+(0.240×CdssC);

wherein R is measured at pH 6-8 in an aqueous carrier; and contacting a malodor with said composition.

### DETAILED DESCRIPTION OF THE INVENTION

The present invention relates to a composition comprising a polyamine polymer and a perfume mixture having a perfume aldehyde component, wherein the perfume aldehydes are compatible with the polyamine polymer when used in formulations to provide superior malodor reduction to treated surfaces and in the air.

As used herein “compatible” or “compatibility” means that the reactivity level of the perfume aldehyde(s) with the polyamine polymer is such that the combination provides malodor reduction in an aqueous formulation. “Compatible” perfume raw materials include perfume aldehydes that are non-reactive (i.e. do not bind with polyamine polymers) as well as perfume aldehydes than are reactive with polyamine polymers but used in limited amounts such that the combination with a polyamine polymer still provides malodor reduction.

### Polyamine Polymer Compatible Perfumes

The compatibility of perfume raw materials (“PRMs”) with polyamine polymers can be analytically determined using the following equation:

R=head space of aqueous composition containing polyamine/head space of aqueous composition nil polyamine polymer

It has been found that perfume aldehydes of the present invention that are non-reactive with polyamine polymers can now be determined by using a specific linear regression equation that predicts R. Linear regression models for designing consumer products are disclosed in US 2012/0101862. Using linear regression models and statistical analysis of experimentally derived data, the equation of the present invention, found to predict R, was derived and is shown below.

R=2.21+(0.159×nrbond)−(0.0559×rvalCar)+(14.4×xch6)+(0.240×CdssC)

R is computed using selected descriptors from a software program called “winMolconn” version 1.1.2.1 (available from Hall Associates Consulting of Quincy, Mass.) and structures are prepared using a 2D connection table (SDF format or SMILES). The following table describes the terms in the equation used in the present invention:

| Descriptor | Coefficient |
|---|---|
| Nrbond | 0.159 |
| rvalCar | −0.0559 |
| xch6 | 14.4 |
| CdssC | 0.240 |
| Y-intercept | 2.21 |

There are three major trends explained by the equation used in the present invention. The first trend focuses on PRMs with rings. Such PRMs tend to have larger surface areas. As a result, they may allow fewer amines to interact with these PRMs due to steric effects with the PRM-polyamine polymer complex. These PRMs are more likely to be compatible with polyamine polymer containing products.

Suitable PRMs that fit this trend are heliotropin, nonaldehyde, p-anisaldehyde, and tetrahydrogeranial. Heliotropin and p-anisaldehyde have greater R values because of their rings. The latter two have lower R values because there are no rings.

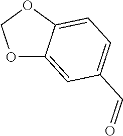 [SMILES: O=Cc1ccc2c(c1)OCO2]

### Heliotropin

Obs. Lupamin™ Ratio=1.65
Pred. Lupamin™ Ratio=1.77

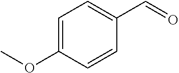

p-Anisaldehyde
Obs. Lupamin™ Ratio=1.52
Pred. Lupamin™ Ratio=1.56

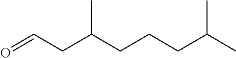

### Tetrahydrogeranial

Obs. Lupamin™ Ratio=0.42
Pred. Lupamin™ Ratio=0.44

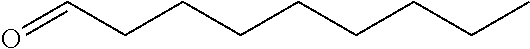

### Nonaldehyde

Obs. Lupamin™ Ratio=0.21
Pred. Lupamin™ Ratio=0.46.

The second trend in the equation focuses on exceptions to the first trend. PRMs that lack rings but are larger in molecular volume and have more rotatable bonds will have a higher R. Conversely, PRMs with rings and less flexibility will have a lower R.

Examples of PRMs that are better in this trend as opposed to the previous trend are adoxal and koavone.

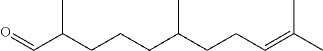

### Adoxal

Obs. Lupamin™ Ratio=0.79
Pred. Lupamin™ Ratio=0.65

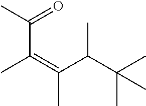

### Koavone

Obs. Lupamin™ Ratio=1.17
Pred. Lupamin™ Ratio=1.11

Examples of PRMs that are inferior in this trend are shown below:

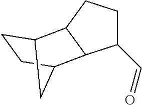 [SMILES: O=CC1CCC2C3CCC(C3)C12]

### Melozone

Obs. Lupamin™ Ratio=0.28
Pred. Lupamin™ Ratio=0.20

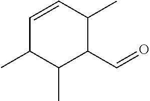

### Isocyclocitral

Obs. Lupamin™ Ratio=0.27
Pred. Lupamin™ Ratio=0.51.

The third trend explains exceptions to the second trend. In this trend, PRMs that are larger and have more flexibility are better than expected using the second trend if they have less double bonds and/or more polarity.

Examples of PRMs that have higher R values in this trend as opposed to the second trend are:

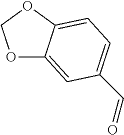 [SMILES: O=Cc1ccc2c(c1)OCO2]

### Heliotropin

Obs. Lupamin™ Ratio=1.65
Pred. Lupamin™ Ratio=1.77

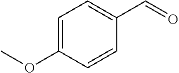

p-Anisaldehyde
Obs. Lupamin™ Ratio=1.52
Pred. Lupamin™ Ratio=1.56.

PRMs that have a lower R in this trend as opposed to the second trend are:

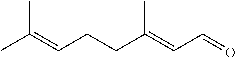

### Citral

Obs. Lupamin™ Ratio=0.23
Pred. Lupamin™ Ratio=0.42

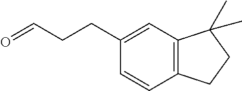

### Hivernal

Obs. Lupamin™ Ratio=0.17
Pred. Lupamin™ Ratio=0.25.

Using the linear regression equation disclosed herein, suitable perfume aldehydes are listed in Table 1.

TABLE 1

| | | Calculated R |
|---|---|---|
| PRM | CAS # | value |
| Cinnamicaldehyde | 104-55-2 | 0.65 |
| Adoxal | 141-13-9 | 0.66 |
| p-Tolualdehyde | 104-87-0 | 0.67 |
| m-Methylbenzaldehyde | 620-23-5 | 0.68 |
| Cuminaldehyde | 122-03-2 | 0.70 |
| Valeraldehyde | 110-62-3 | 0.71 |
| p-Isopropylphenylacetaldehyde | 4395-92-0 | 0.71 |
| 4-tert-Butylbenzaldehyde | 939-97-9 | 0.72 |
| 5-Methylfurfural | 620-02-0 | 0.72 |
| p-t-butylphenylacetaldehyde | 109347-45-7 | 0.75 |
| alpha,4-Dimethylbenzenepropana | 41496-43-9 | 0.75 |
| 3-(p-Isopropylphenyl)propional | 7775-00-0 | 0.75 |
| Bourgeonal | 18127-01-0 | 0.80 |
| Florhydral | 125109-85-5 | 0.84 |
| Cymal | 103-95-7 | 0.85 |
| Floralozone | 67634-15-5 | 0.90 |
| Lilial | 80-54-6 | 0.91 |
| Florazon(ortho-isomer) | 67634-14-4 | 0.91 |
| Mefloral | 62518-65-4 | 0.92 |
| Phenylacetaldehyde | 122-78-1 | 0.92 |
| Silvial | 6658-48-6 | 0.93 |
| 2,4-Dimethyl-3-Cyclohexene-1-c | 68039-49-6 | 0.93 |
| Vertoliff | 27939-60-2 | 0.93 |
| Veltonal | 68555-62-4 | 0.94 |
| Scentenal | 86803-90-9 | 0.95 |
| Benzaldehyde | 100-52-7 | 0.96 |
| Trifernal | 16251-77-7 | 0.96 |
| Hydratropaldehyde | 93-53-8 | 0.97 |
| alpha-methylcinnamaldehyde | 101-39-3 | 0.98 |
| Scentenal | 193425-86-4 | 0.99 |
| 3-Methyl-5-phenyl-1-pentanal | 55066-49-4 | 1.05 |
| Perillaldehyde | 2111-75-3 | 1.07 |
| cis-3-Hexenyloxyacetaldehyde | 68133-72-2 | 1.08 |
| Acalea | 84697-09-6 | 1.12 |
| Limonenal | 6784-13-0 | 1.13 |
| Cetonal | 65405-84-7 | 1.17 |
| Boronal | 3155-71-3 | 1.19 |
| PrecyclemoneB | 52474-60-9 | 1.23 |
| Isohexenylcyclohexenylcarboxal | 37677-14-8 | 1.27 |
| o-Methoxycinnamaldehyde | 1504-74-1 | 1.29 |
| alpha-Amylcinnamaldehyde | 122-40-7 | 1.32 |
| Citronellyloxyacetaldehyde | 7492-67-3 | 1.32 |
| 2-Phenyl-3-(2-furyl)prop-2-ena | 57568-60-2 | 1.34 |
| Methoxycitronellal | 3613-30-7 | 1.35 |
| Methoxymelonal | 62439-41-2 | 1.36 |
| Hexylcinnamicaldehyde | 101-86-0 | 1.40 |
| 7-Ethoxy-3,7-dimethyloctanal | 3613-33-0 | 1.43 |
| Pinoacetaldehyde | 30897-75-7 | 1.49 |
| 6,6-dimethyl-2-norpinene-2-pro | 33885-51-7 | 1.49 |
| Myrtenal | 564-94-3 | 1.53 |
| p-Anisaldehyde | 123-11-5 | 1.56 |
| CyclemoneA | 68991-97-9 | 1.57 |
| Canthoxal | 5462-06-6 | 1.59 |
| o-Anisaldehyde | 135-02-4 | 1.59 |
| 4-Ethoxybenzaldehyde | 10031-82-0 | 1.61 |
| 2-Ethoxybenzaldehyde | 613-69-4 | 1.65 |
| alpha,alpha,6,6-tetramethylbic | 33885-52-8 | 1.66 |
| Helional | 1205-17-0 | 1.74 |
| Heliotropin | 120-57-0 | 1.77 |
| Hydroxycitronellal | 107-75-5 | 1.90 |
| Veratraldehyde | 120-14-9 | 2.07 |
| 4-hydroxy-3-methoxy-cinnamalde | 458-36-6 | 2.20 |
| Maceal | 67845-30-1 | 2.21 |
| Vanillin | 121-33-5 | 2.52 |
| 2,4,5-trimethoxy-benzaldehyde | 4460-86-0 | 2.52 |
| 3,4,5-trimethoxybenzaldehyde | 86-81-7 | 2.52 |
| 2,4,6-trimethoxybenzaldehyde | 830-79-5 | 2.53 |
| 2,3,4-trimethoxy-benzaldehyde | 2103-57-3 | 2.53 |
| Ethylvanillin | 121-32-4 | 2.57 |
| Lyral | 31906-04-4 | 2.61 |
| Vanillinacetate | 881-68-5 | 3.15 |
| Ethylvanillinacetate | 72207-94-4 | 3.23 |
| Vanillinisobutyrate | 20665-85-4 | 3.28 |

Compositions of the present invention may comprise from about 0.001% to about 10%, or from about 0.001% to about 5%, or from about 0.001% to about 3%, or from about 0.01% to about 1%, or from about 0.05% to about 1.0%, by weight of said composition, of a perfume mixture.

The perfume mixture may comprise a perfume aldehyde component comprising less than about 85%, or less than about 80%, or less than about 70%, or less than 65%, or less about 60%, by weight of the perfume aldehyde component, of R less than 1 perfume aldehydes. In some embodiments, the perfume mixture may comprise a perfume aldehyde component comprising less than about 85%, by weight of said perfume aldehyde component, of perfume aldehydes having an R value of less than 0.8, or about 0.5 to less than 1, or from about 0.1 to less than 1. Alternatively, the perfume mixture may comprise a perfume aldehyde component comprising upto 100%, or from about 30% to about 100%, or from about 75% to about 100%, by weight of the perfume aldehyde component, of perfume aldehydes having an R value greater than 0.8, or greater than greater than 1, or from about 0.8 to about 4.

In one embodiment, the perfume aldehydes may include one or more of the following: amyl cinnamic aldehyde, anisic aldehyde, benzaldehyde, citronellal, cuminic aldehyde, citronellal oxyacetaldehyde, floralozone, heliotropin, and hexyl cinnamic aldehyde. In another embodiment, at least 10%, or at least 15% or at least 20%, or at least 25%, or at least 30% of the perfume mixture comprises one or more of the following perfume aldehydes: amyl cinnamic aldehyde, anisic aldehyde, benzaldehyde, citronellal, cuminic aldehyde, citronellal oxyacetaldehyde, floralozone, heliotropin, and hexyl cinnamic aldehyde.

Additional perfume materials may be included in the perfume mixture. Suitable perfume materials may include alcohols, (e.g. phenyl ethyl alcohol, octanol, linalool, etc.); esters (e.g. hexyl acetate, ethyl acetate, geranyl propionate, etc); lactones (e.g. gamma decalactone, nonalactone, etc); ketones/ionones, (e.g. delta damscone, koavone, ionone-gamma methyl, ionone beta, etc); alkanes (e.g. terpiniolenes, terpinenes, isolongafolene, d-limonene, pinenes, etc.).

### Polyamine Polymer

The polyamine polymer of the present invention can be either linear or cyclic. The polyamine polymer has a general formula (I):

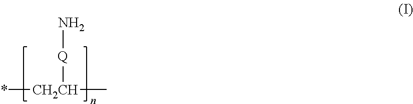

where Q is an integer having values between 0-3.

Non-limiting examples of polyamine polymers include polyvinylamine (“PVam”), polyethyleneimine (“PEI”) that are linear or branched, polyamidoamine (“PAMam”), polyallyamines (“PAam”), polyetheramines (“PEam”) or other nitrogen containing polymers, such as lysine, or mixtures of these nitrogen containing polymers.

a. PVams

In one embodiment, the polyamine polymer includes a PVam backbone. A PVam is a linear polymer with pendent, primary amine groups directly linked to the main chain of alternating carbons. PVams are manufactured from hydrolysis of poly(N-vinylformamide) (PVNF) which results in the conversion of formamide units to amino groups as described by the following formula (Ia):

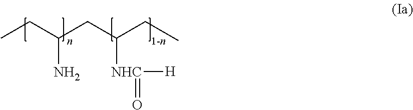

where n is a number from 0.1 to 0.99 depending on the degree of hydrolysis. For instance, in 95% hydrolyzed PVam, n will be 0.95 while 5% of the polymer will have vinylformamide units.

PVams may be partially hydrolyzed meaning that 1% to 99%, alternatively 30% to 99%, alternatively 50% to 99%, alternatively 70% to 99%, alternatively 80% to 99%, alternatively 85% to 99%, alternatively 90% to 99%, alternatively 95% to 99%, alternatively 97% to 99%, alternatively 99% of the PVam is hydrolyzed. It has been found that high degree of hydrolysis of PVam increases the resulting polymer's ability to mitigate the odors.

PVams that can be hydrolyzed may have an average molecular weight (“MW”) of 5,000 to 350,000. Suitable hydrolyzed PVams are commercially available from BASF. Some examples include Lupamin™ 9095, 9030, 5095, and 1595.

Such hydrolyzed PVams may then be hydrophobically modified. Hydrophobic modification, as described below may further improve malodor removal efficacy.

b. Polvalkylenimine/PEIs

In another embodiment, the polyamine polymer includes a polyalkylenimine backbone. Polyalkylenimines include PEIs and polypropylenimines as well as the C4-C12 alkylenimines

PEI is a suitable polyalkylenimine The chemical structure of a PEI follows a simple principle: one amine function and two carbons. PEIs have the following general formula (Ib):

—(CH2-CH2-NH)n- (Ib)

where n=10-105.

PEIs constitute a large family of water-soluble polyamine polymers of varying molecular weight, structure, and degree of modification. They may act as weak bases and may exhibit a cationic character depending on the extent of protonation driven by pH.

PEIs are produced by the ring-opening cationic polymerization of ethyleneimine as shown below.

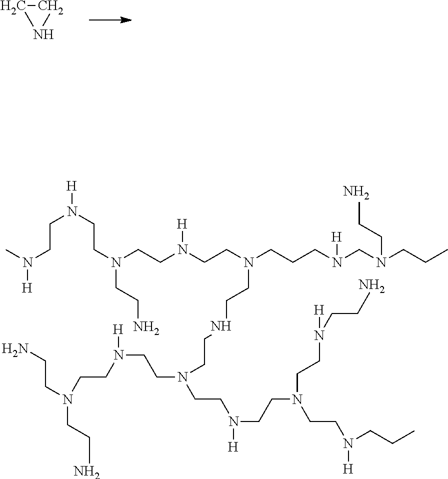

PEIs are believed to be highly branched containing primary, secondary, and tertiary amine groups in the ratio of about 1:2:1. PEIs may comprise a primary amine range from about 30% to about 40%, alternatively from about 32% to about 38%, alternatively from about 34% to about 36%. PEIs may comprise a secondary amine range from about 30% to about 40%, alternatively from about 32% to about 38%, alternatively from about 34% to about 36%. PEIs may comprise a tertiary amine range from about 25% to about 35%, alternatively from about 27% to about 33%, alternatively from about 29% to about 31%.

Other routes of synthesis may lead to products with a modified branched chain structure or even to linear chain PEIs. Linear PEIs contain amine sites in the main chain while the branched PEIs contain amines on the main and side chains. Below is an example of a linear PEI.

### Linear PEI

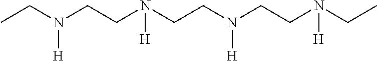

The composition of the present invention may comprise PEIs having a MW of about 800 to about 2,000,000, alternatively about 1,000 to about 2,000,000, alternatively about 1,200 to about 25,000, alternatively about 1,300 to about 25,000, alternatively about 2,000 to about 25,000, alternatively about 10,000 to about 2,000,000, alternatively about 25,000 to about 2,000,000, alternatively about 25,000.

In one embodiment, the PEI may have a specific gravity of 1.05 and/or an amine value of 18 (mmol/g, solid). For clarity, such specific gravity and/or amine value of the PEI describes the PEI before it is modified or added as part of an aqueous composition. One skilled in the art will appreciate, for example, the primary and secondary amino groups may react with other components of the composition.

Exemplary PEIs include those that are commercially available under the tradename Lupasol® from BASF or the tradename Epomine™ from Nippon Shokubia.

In some embodiments, less than 100% of the active amine sites are substituted with hydrophobic functional groups, alternatively about 0.5% to about 90%, alternatively about 0.5% to about 80%, alternatively about 0.5% to about 70%, alternatively about 0.5% to about 60%, alternatively about 0.5% to about 50%, alternatively about 0.5% to about 40%, alternatively about 0.5% to about 35%, alternatively about 0.5% to about 30%, alternatively about 1% to about 30%, alternatively about alternatively about 1% to about 25%, alternatively about 1% to about 20%, alternatively about 5% to about 20%, alternatively about 10% to about 30%, alternatively about 20% to about 30%, alternatively about 20% of the active amine sites are substituted with hydrophobic functional groups. When a PEI has active amine sites that are fully substituted with hydrophobic functional groups, such hydrophobically modified PEI may have no activity for malodor control.

c. PAMams

In another embodiment, the polyamine polymer includes a PAMam backbone. PAMams are polymers whose backbone chain contains both amino functionalities (—NH) and amide functionalities (—NH—C(O)). PAMams also contain primary amine groups and/or carboxyl groups at the termini of polymer chain. The general structure of a PAMam is below (Ic).

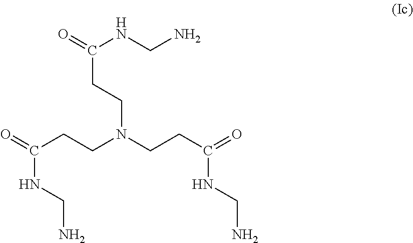 [SMILES: NCNC(=O)CCN(CCC(=O)NCN)CCC(=O)NCN]

d. PAams In another embodiment, the polyamine polymer includes a PAam backbone. PAams are prepared from polymerization of allyamine-C₃H₅NH2. Unlike PEIs, they contain only primary amino groups that are linked to the side chains. The general formula for a PAAm is shown below (I1d).

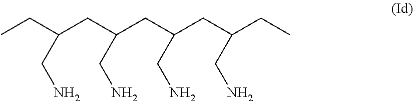

e. PEams

In yet another embodiment, the polyamine polymer includes a PEam backbone. PEams contain a primary amino groups attached to the end of a polyether backbone. The polyether backbone may be based on propylene oxide (“PO”), ethylene oxide (“EO”), or mixed PO/EC). The general formula for a PEam is shown below (Ie).

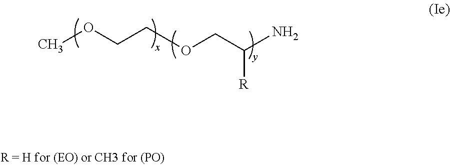

These so-called monoamines, M-series, are commercially available from Hunstman under the tradename Jeffamine® monoamines In another embodiment, the polyamine polymer may include a PEam backbone having diamines as shown below (Ie1).

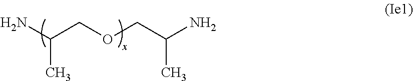

Diamines are commercially available from Hunstman under the tradename Jeffamine® diamines (e.g. D, ED, and EDR series). The polyamine polymer may also include a PEam backbone having triamines (e.g. Jeffamine® triamine T-series).

f. Dendrimers

A further class of amine compounds is the class of dendrimers. Suitable dendrimers carry free primary amine groups at the periphery of the spherical molecules.

By dendrimers, it is understood that the molecule is built up from a core molecule as described, e.g., in WO 96/02588, in Synthesis (February 1978, pgs. 155-158), or in Encyclopedia of Polymer Science & Engineering, 2^{nd} ed. (Hedstrand et al., in particular pgs. 46-91). The core is typically connected to multifunctional components to build up the “generations”. For the purpose of the present invention, the nature of the inner generations is not critical. They can be based on e.g. polyamidoamines, polyamidoalcohols, polyethers, polyamides, polyethylenimines, etc. The outer generation(s) contain accessible primary amino functions.

Also suitable are the glyco dendrimers as described in, e.g., Nachrichten aus Chemie 11 (1996, pgs. 1073-1079); and in WO 97/48711 provided that free primary amine groups are present at the surface of these molecules.

Preferred dendrimer are the polyethylenimine and/or polypropylenimine dendrimers; the commercially available Starburst® polyamidoamine dendrimers, generation G0-G10 from Dendritech; and the dendrimers Astromols®, generation 1-5 from DSM being DiAminoButane polyamine polymers DAB (PA)x dendrimers with x=2ⁿ×4 and n being generally comprised between 0 and 4.

h. Hydrophobic Modification

Hydrophobic modification of the polyamine polymers disclosed herein may improve perfume aldehyde compatibility. As such, the composition of the present invention may also include a hydrophobically modified polyamine polymer (“HMP”) as described in US 2012/0183488A1. A HMP is formed from a polyamine polymer having a primary, secondary, and/or tertiary amine group that is modified with a hydrophobic group such as an alkyl, alkenyl, alkyloxide, or amide. The hydrophobic group of the HMP may be linear, branched, or cyclic alkyl, hydroxyalkyl, alkenyl, hydroxyalkenyl, alkyl carboxyl, alkyloxide, alkanediyl, amide, or aryl. Although the amine group has been modified, a HMP has at least one free and unmodified primary, secondary, and/or tertiary amine group, to react with malodorous components. Not wishing to be bound by theory, hydrophobic modification may increase a polymer's affinity for hydrophobic odors, thus enabling interactions between the odor molecules and active amine sites. In turn, HMPs may improve the breadth of malodor removal efficacy.

HMPs of the present invention have the general formula (Ih):

P(C)x (Ih)

wherein:

P is a polyamine polymer;

C is a C2 to C26 hydrophobic group; and

x is the total degree of substitution, which is less than 100%, of amine sites on the polymer.

In some embodiments, the hydrophobic group is a C2 to C12, alternatively a C2 to C10, alternatively a C4 to C10, alternatively a C16 to C26, alternatively a C6. Where cyclodextrin is included in a formulation, it may be desirous to use a HMP that has been modified with a C2 to C10 alkyl group, alternatively a C16 to C26 alkyl group, alternatively a C6 alkyl group, since such alkyl groups are cyclodextrin compatible.

Polyamine polymers suitable for use in the present invention are water-soluble or dispersible. In some embodiments, the primary, secondary, and/or tertiary amines of the polyamine polymers chain are partially substituted rendering hydrophobicity while maintaining the desired water solubility. The minimum solubility index of a polyamine polymer may be about 2% (i.e. 2 g/100 ml of water). A suitable polyamine polymer for an aqueous fabric refresher formulation may have a water solubility percentage of greater than about 0.5% to 100%, alternatively greater than about 5%, alternatively greater than about 10%, alternatively greater than about 20%.

The water solubility index can be determined by the following test.

### Water Solubility

This test illustrates the benchmarking ambient temperature water solubility of polyamine polymers against beta-cyclodextrin (1.8 g/100 ml) and hydroxypropyl modified beta cyclodextrin (60+g/100 ml). 1% water solubility is used as a screening criteria for polyamine polymers suitable for use in aqueous fabric refresher formulations.

Room temperature equilibrium water solubility of polymers may be determined by adding weighed quantities of polymers into 100 ml of deionized water and allowing the added polymers to completely dissolve. This process is repeated until the added polymers are no longer soluble. Equilibrium water solubility is then calculated based on how much polymer is dissolved in 100 ml water.

| | Equilibrium |
|---|---|
| | Water Solubility |
| Polymer | (g/100 ml water at 25° C.) |
| Lupasol G100 (PEI 5,000) | miscible at all levels (70+) |
| C6 modified PEI 1800 | 30+ |
| (0.25 C6/NH) | |
| Dodecene oxide modified PEI5,000 | ~24 |
| (0.1 dodecene oxide/NH) | |
| Dodecene oxide modified PEI5,000 | ~4 |
| (0.2 dodecene oxide/NH) | |
| Dodecene oxide modified PEI5,000 | <0.1 |
| (0.5 dodecene oxide/NH) | |
| Dodecene oxide modified PEI25,000 | ~21 |
| (0.1 dodecene oxide/NH) | |
| Dodecene oxide modified PEI25,000 | <0.1 |
| (0.2 dodecene oxide/NH) | |
| Dodecene oxide and EO modified PEI25,000 | ~6 |
| (0.8 EO and 0.2 dodecene oxide/NH) | |

When the polyamine polymer is not water soluble (e.g. less than 0.05%), capping with a hydrophilic molecule may be desired to assist with water solubility. Suitable hydrophilic molecules include EO or other suitable hydrophilic functional groups.

Suitable levels of polyamine polymers in the present composition are from about 0.001% to about 10%, alternatively from about 0.001% to about 2%, alternatively from about 0.01% to about 1%, alternatively from about 0.01% to about 0.8%, alternatively from about 0.01% to about 0.6%, alternatively from about 0.01% to about 0.1%, alternatively from about 0.01% to about 0.07%, alternatively about 0.07%, alternatively about 0.5%, by weight of the composition. Compositions with higher amount of polyamine polymer may make fabrics susceptible to soiling and/or leave unacceptable visible stains on fabrics as the composition evaporates off of the fabric.

### Method of Use

The polyamine polymer compatible perfume materials can be formulated into a variety of products such as fabric refreshers, air fresheners, hand and automatic dishwashing formulas, liquid laundry detergents, hard surface cleaning formulas, and the like.

Also provided herein is a method for reducing malodor comprising the step of providing an aqueous composition comprising effective amounts of a polyamine polymer and a perfume aldehyde component and contacting by means of spraying or spreading the composition on a surface or in the air. By “surface”, it is meant any surface onto which the compound can deposit. Typical examples of such material are fabrics, hard surfaces such as dishware, floors, bathrooms, toilet, kitchen, garbage/trash bags, and other surfaces in need of a malodor reduction.

### EXAMPLES

### Example 1

### Fabric Refresher Formulas

Polyamine polymer perfume interactions were studied in aqueous fabric refresher formulations. Aqueous formulations containing a perfume mixture and a polyamine polymer was prepared according to Table 2 and studied against a control fabric refresher formulation without a polyamine polymer. The perfume mixture was prepared as shown in Table 3 and used in the formulations given in Table 2. Studies were conducted using 500 ppm (0.05%) polyamine polymer and perfume mixtures at 2 levels (500 ppm (0.05%) and 10,000 ppm (0.1%)).

TABLE 2
Aqueous Fabric Refresher Formulations

| Ingredient | Control | Test Formulation |
|---|---|---|
| Deionized water | Balance | Balance |
| Ethanol | 3% | 3% |
| Surfactant | 0.1% | 0.1% |
| (Silwet L-7600) | | |
| Diethyleneglycol | 0.175% | 0.175% |
| Alkylammonium chloride | 0.06% | 0.06% |
| (Uniquat 2250) | | |
| Preservative | — | — |
| (Koralone B-119) | | |
| Perfume mixture comprising 0.1 wt % | 0.15% | 0.15% |
| of a customized perfume mixture | | |
| and 0.05% emulsifier (Basophor | | |
| ELH60) | | |
| Polyamine Polymer | — | 0.05% |
| Maleic acid (30%) | as needed | as needed |
| Final pH | 6.8 | 6.8 |

TABLE 3
Perfume Mixture

| Material Name | Percent |
|---|---|
| Hexyl cinnamic aldehyde | 3.947 |
| Adoxal | 3.839 |
| Dupical | 3.728 |
| Lyral | 3.838 |
| 2-Tridecenal | 3.583 |
| Lauric_aldehyde | 3.363 |
| Methyl_nonyl_acetaldehyde | 3.363 |
| 4-tert-butylbenzaldehyde | 2.960 |
| Dihydrocitronellal | 2.851 |
| Citral | 2.778 |
| Citronellal | 2.815 |
| Isocyclocitral | 2.778 |
| 2,4,6-Trimethoxybenzaldehyde | 3.580 |
| Cuminic_aldehyde | 2.704 |
| 2-Methyloctanal | 2.596 |
| Para Tolyl aceatladehyde | 2.448 |
| o-Anisaldehyde | 2.484 |
| Anisic aldehyde | 2.484 |
| Vanillin | 2.776 |
| Hexyl aldehyde | 1.828 |
| 2-Methylpenanal | 1.828 |
| Benzaldehyde | 1.936 |
| Trans-2_Hexenal | 1.791 |
| nonyl aldehyde | 2.596 |
| lauric aldehyde | 3.363 |
| myrstic aldehyde | 3.875 |
| beta ionone | 3.509 |
| delta Damascone | 3.509 |
| koavone | 3.327 |
| Tabanone Coeur | 3.472 |
| cashmeran | 3.765 |
| zingerone (Vanillyl acetone) | 3.544 |
| L-carvone | 2.741 |
| | 100.000 |

### Example 2

### Perfume GC-MS Analysis and Selection of Reacting and Non-reacting Aldeyhdes

GC-MS was used to study polymer perfume aldehyde interactions using the formulations prepared in Example 1. For the GC-MS analysis, 2 ml of each sample was transferred into 20 ml headspace vial, equilibrated at room temperature for 1 hr, and incubated at 40° C. for 20 minutes. Then, 1 ml of head space was injected into GC-MS using 1 to 10 split, and perfume raw material intensities were measured. Perfume raw material peak area ratios were then calculated using the control sample without a polyamine polymer. An example of perfume raw material peak area ratios of solutions with a polyamine polymer is shown in Table 4.

TABLE 4
GC-MS head space data with Lupamin ™
1595 normalized to Control.

| | | Polyamine polymer |
|---|---|---|
| Material Name | Control | (Lupamin 1595) |
| 2-methyl petanal | 1.000 | 0.864 |
| Hexanal | 1.000 | 0.222 |
| Trans-2-hexenal | 1.000 | 0.226 |
| Methyl Octyl acetaldehyde | 1.000 | 0.836 |
| Dihydrocitronellal | 1.000 | 0.415 |
| Nonanal | 1.000 | 0.209 |
| Citronellal | 1.000 | 2.597 |
| Kaovone | 1.000 | 1.185 |
| Kaovone | 1.000 | 1.159 |
| 2-acetyl thiophene | 1.000 | 0.794 |
| Isocyclocitral n | 1.000 | 0.271 |
| Benzaldehyde | 1.000 | 1.030 |
| Kaovone | 1.000 | 1.171 |
| Methyl Nonyl Acetaldehyde | 1.000 | 0.772 |
| Neral | 1.000 | 0.261 |
| Tridecenal | 1.000 | 0.470 |
| Carvone | 1.000 | 1.103 |
| Delta-damascone | 1.000 | 1.228 |
| Cumin Aldehyde | 1.000 | 0.977 |
| Adoxal | 1.000 | 0.751 |
| 1,4-diacety lbenzene | 1.000 | 0.913 |
| Beta Ionone | 1.000 | 1.222 |
| Dupical | 1.000 | 0.932 |
| Lauric Aldehyde | 1.000 | 0.169 |
| Anisic Aldehyde | 1.000 | 1.521 |
| Hexyl Cinnamic Aldehdye | 1.000 | 1.480 |

PRMs that react with polyamine polymer have a headspace concentration less than 1.00 with respect to the normalized Control.

### Example 3

### Polyamine Polymer Malodor Control Performance in the Presence of Reacting or Non-Reacting Perfume Aldeyhdes

This example illustrates malodor control performance of polyamine polymers for greasy odors in the presence of non-reacting and reacting perfume aldehydes. Hydrophobic greasy cooking odors were represented by aldehydes such as nonanal. Octanal and hexylcinnamic aldehyde were used as reactive and non-reactive perfume aldehydes, respectively. Aqueous solutions of Lupamin™ 1595 polymer and octanal or hexylcinnamic aldeydes were prepared according to Table 5. Aldeyhdes were emulsified with Basophor/Aquasolved and added into the Lupamin 1595 solution at pH6.8 with maleic acid. Lupamin™ 1595 was used at 0.052% and concentrations of octanal and hexylcinnamic aldeydes were varied from low to high to represent Lupamin modification between 20% and 90%.

TABLE 5

| | Percent by weight (wt. %) | | |
|---|---|---|---|
| Formula Ingredient | Control 1 | Control 2 | Test Solution |
| Water | Balance | Balance | Balance |
| Surfactant (Silwet ™ L7600) | 0.1 | 0.1 | 0.1 |
| Ethanol | 3 | 3 | 3 |
| Lupamin ™ 1595 | — | — | 0.05 |
| Octanal or hexylcinnamic | — | 0.03-0.23 | 0.03-0.23 |
| aldeyde | | | |
| Emulsifier | As needed | As needed | As needed |
| (Basophor ™/Aquasolved ™) | | | |
| Maleic acid (30%) | As needed | As needed | As needed |
| Final pH | 7 | 7 | 7 |

5 ml solution prepared according to Table 5 was transferred into 20 ml headspace vial and spiked with 0.5 microliter of nonanal. Then, the mixture was equilibrated at room temperature for 1 hour and incubated at 30° C. for 40 minutes. Finally, 1 ml of head space was injected into GC-MS and nonanal intensities were measured via SPME. Reductions in nonanal head space levels was then normalized to Control #1 and reported in Table 6. High levels of nonanal reduction are attributed to high malodor control efficacy of polymers. Table 6 demonstrates that compositions having a perfume mixture with upto 100% of R greater than 1 perfume aldehydes do not affect malodor efficacy of polymers since these perfume aldehydes are non-reacting aldehydes. On the other hand, compositions having a perfume mixture comprising more than 80% of R less than 1 perfume aldehydes have less malodor efficacy than the same composition having less than 80% of R less than 1 perfume aldehydes.

TABLE 6

| | % Nonanal |
|---|---|
| Sample | Reduction |
| Control #1 (nil polymer/nil octanal/nil hexylcinnamic | 0 |
| aldehyde) | |
| Control #2 (polymer only) | 100 |
| (0.052% Lupamin + 0.03% octanal) | 99 |
| (0.052% Lupamin + 0.065% octanal) | 99 |
| (0.052% Lupamin + 0.09% octanal) | 99 |
| (0.052% Lupamin + 0.11% octanal) | 50 |
| (0.052% Lupamin + 0.13% octanal) | 20 |
| (0.052% Lupamin + 0.05% hexylcinnamic aldeyhde) | 100 |
| (0.052% Lupamin + 0.12% hexylcinnamic aldeyhde) | 100 |
| (0.052% Lupamin + 0.23% hexylcinnamic aldeyhde) | 100 |

“Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.”

“While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.”

1-20. (canceled)

21. A malodor control composition comprising:
about 0.001% to about 2%, by weight of said composition, of a polyvinylamine polymer; and
a perfume mixture comprising a perfume aldehyde component comprising less than about 85%, by weight of said perfume aldehyde component, of perfume aldehydes having an R value less than 1 wherein:
R=2.21+(0.159×nrbond)−(0.0559×rvalCar)+(14.4×xch6)+(0.240×CdssC);
wherein R is measured at pH 6-8 in an aqueous carrier.

22. The composition of claim 21, wherein said R value is about 0.5 to less than 1.

23. The composition of claim 21, wherein said perfume aldehyde component comprises less than about 70%, by weight of said perfume aldehyde component, of perfume aldehydes having an R value less than 1.

24. The composition of claim 21, wherein said perfume mixture comprises at least one perfume aldehyde selected from the group consisting of: amyl cinnamic aldehyde, anisic aldehyde, benzaldehyde, citronellal, cuminic aldehyde, citronellal oxyacetaldehyde, floralozone, heliotropin, hexyl cinnamic aldehyde, and mixtures thereof.

25. The composition of claim 21 wherein said perfume mixture comprises at least 10%, by weight of said perfume mixture, of at least one perfume aldehyde selected from the group consisting of: amyl cinnamic aldehyde, anisic aldehyde, benzaldehyde, citronellal, cuminic aldehyde, citronellal oxyacetaldehyde, floralozone, heliotropin, hexyl cinnamic aldehyde, and mixtures thereof.

26. The composition of claim 21 wherein said perfume mixture comprises hexylcinnamic aldehyde and anisic aldehyde.

27. The composition of claim 21, wherein said polyamine polymer is present in an amount from about 0.05% to about 0.1%, by weight of said composition.

28. The composition of claim 21, wherein said polyvinylamine polymer is a hydrophobically modified polyvinylamine polymer.

29. The composition of claim 21, wherein said aqueous carrier is present in an amount of greater than about 70%, by weight of said composition.

30. The composition of claim 21, further comprising less than 3%, by weight of said composition, of a surfactant.

31. The composition of claim 21, wherein said pH is 7.

32. The composition of claim 21, wherein said composition is substantially free of anionic surfactants.

33. A malodor control composition comprising:
about 0.001% to about 2%, by weight of said composition, of a polyvinylamine polymer; and
a perfume mixture comprising a perfume aldehyde component comprising:
at least one perfume aldehyde selected from the group consisting of: amyl cinnamic aldehyde, anisic aldehyde, benzaldehyde, citronellal, cuminic aldehyde, citronellal oxyacetaldehyde, floralozone, heliotropin, hexyl cinnamic aldehyde, and mixtures thereof; and
less than about 60%, by weight of said perfume aldehyde component, of perfume aldehydes having an R value less than 1 wherein:
R=2.21+(0.159×nrbond)−(0.0559×rvalCar)+(14.4×xch6)+(0.240×CdssC);
wherein R is measured at pH 6-8 in an aqueous carrier.

34. The composition of claim 33 wherein said at least one perfume aldehyde comprises hexylcinnamic and anisic aldehyde.

35. The composition of claim 33, wherein said at least one perfume aldehyde comprises hexyl cinnamic aldehyde, anisic aldehyde, citronellal, cuminic aldehyde, and benzaldehyde.

36. The composition of claim 33 wherein said perfume mixture comprises at least 10%, by weight of said perfume mixture, of at least one perfume aldehyde selected from the group consisting of: amyl cinnamic aldehyde, anisic aldehyde, benzaldehyde, citronellal, cuminic aldehyde, citronellal oxyacetaldehyde, floralozone, heliotropin, hexyl cinnamic aldehyde, and mixtures thereof.

37. The composition of claim 33, wherein said polyamine polymer is present in an amount from about 0.05% to about 0.1%, by weight of said composition.

38. The composition of claim 33, further comprising less than 3%, by weight of said composition, of a surfactant.

39. The composition of claim 33, wherein said pH is 7.

40. A composition comprising:
about 0.001% to about 2%, by weight of said composition, of a polyvinylamine polymer; and
a perfume mixture comprising a perfume aldehyde component comprising up to about 100%, by weight of said perfume aldehyde component, of perfume aldehydes having an R value greater than 1, wherein at least one perfume aldehyde is selected from the group consisting of: amyl cinnamic aldehyde, anisic aldehyde, benzaldehyde, citronellal, cuminic aldehyde, citronellal oxyacetaldehyde, floralozone, heliotropin, hexyl cinnamic aldehyde, and mixtures thereof, wherein
R=2.21+(0.159×nrbond)−(0.0559×rvalCar)+(14.4×xch6)+(0.240×CdssC);
wherein R is measured at pH 6-8 in an aqueous carrier.
